(19) United States
(12) Patent Application Publication
(10) Pub. No.: US 2024/0118205 A1
(43) Pub. Date: Apr. 11, 2024

(54) BACTERIAL CELLULOSE-BASED BIOSENSOR AND USE THEREOF

(71) Applicant: Jiangnan Univesity, Wuxi (CN)

(72) Inventors: Lingfeng LONG, Wuxi (CN); Fubao Sun, Wuxi (CN); Yun Hu, Wuxi (CN); Le Xie, Wuxi (CN)

(21) Appl. No.: 18/527,123

(22) Filed: Dec. 1, 2023

Related U.S. Application Data
(63) Continuation of application No. PCT/CN2022/122046, filed on Sep. 28, 2022.

(30) Foreign Application Priority Data
Oct. 12, 2021 (CN) 202111185375.6

(51) Int. Cl. G01N 21/64; C12N 15/70

(52) U.S. Cl. CPC G01N 21/643; C12N 15/70

(57) ABSTRACT

The invention provides a bacterial cellulose-based biosensor, including bacterial cellulose (BC) and a cell presenting a cellulose-binding module CBM2a on the surface. The cell is attached to BC through CBM2a. The cell expresses CBM2a by using pETDuet-tac as a vector. The pETDuet-tac is obtained by replacing two T7 promoters on the vector pETDuet by tac promoters, that is, an upstream first tac promoter and a downstream second tac promoter. The pETDuet-tac includes a gene encoding a fluorescent protein downstream of the first tac promoter and a gene encoding CBM2a presented on the surface downstream of the second tac promoter. By the BC-based biosensor, efficient and specific immobilization of cells on the BC matrix is enabled, the biological activity of cells is maintained, the fluorescence signal output is enhanced, and sufficient pores are provided for the entry and exit of a detected substance, thereby significantly improving the detection sensitivity.

This application is a Continuation Application of PCT/CN2022/122046, filed on Sep. 28, 2022, which claims priority to Chinese Patent Application No. 202111185375.6, filed on Oct. 12, 2021, which is incorporated by reference for all purposes as if fully set forth herein.

A Sequence Listing XML file named “10015_0139.xml” created on Dec. 1, 2023, and having a size of 12,828 bytes, is filed concurrently with the specification. The sequence listing contained in the XML file is part of the specification and is herein incorporated by reference in its entirety.

### FIELD OF THE INVENTION

The present invention relates to the fields of molecular biology, genetic engineering, nanomaterials and biosensors, and in particular to a bacterial cellulose-based biosensor and use thereof.

### DESCRIPTION OF THE RELATED ART

Biosensors based on fluorescence detection play an important role in environmental pollutant detection, biochemical diagnosis, biomedical sensing, and other areas. Up to now, many engineered strains have been reported as sensors to detect chemicals such as heavy metals, organic compounds and antibiotics. However, during the use of biosensors, an engineered strain is required to be immobilized on a material platform to maintain the biological activity of cells and enhance the fluorescence signal output. In addition, a desirable biosensor platform needs to have enough pores for the entry and exit of a tested substance, and minimize the environmental pollution at the same time.

Bacterial cellulose (BC) refers to cellulose synthesized by some microorganisms such as Acetobacter, Agrobacterium, Rhizobium and Sarcina under various conditions. Because of its good biocompatibility, high mechanical strength, powerful water retention capacity, high porosity and other characteristics, BC has broad application potential as a biosensor platform. BC is widely used in gas sensors, surface acoustic wave humidity sensors, and electrochemical sensors, etc. However, when it is used in biosensors, because of the large pores between BC fibers, bacterial cells cannot be attached thereto for a long period of time, and will escape during the sensing process, which limits the use of BC as a biosensor.

A bacterial sensor based on P(HEMA-co-HAETC) hydrogel is developed by Kim et al. (2019). The bacterial sensor is prepared by preparing P(HEMA-co-HAETC) hydrogel beads by electrospray, and loading cells on the hydrogel beads through 12 hrs of incubation. The preparation process is very time-consuming and requires the use of professional equipment. A method for embedding cells in a BC material by co-incubating recombinant cells with a BC producing strain (Gluconacetobacter xylinus) is proposed by Drachuk et al. (2017). However, in this method, the BC producing strain is introduced into the biosensor system, which interferes with the detection of an analyte. In addition, an immobilization method for enhancing the affinity of negatively charged bacterial cells to a carrier by modifying porous polyacrylamide microspheres to form positive charges is proposed by Yoetz Kopelman et al. (2016). Although this method is proved to be feasible as a whole-cell biosensor, it causes nonspecific binding between cells and matrix.

### SUMMARY OF THE INVENTION

To solve the above technical problems, the present invention discloses a bacterial cellulose-based biosensor, which enhances the adhesion of cells to bacterial cellulose by presenting cellulose-binding module (CBM) as an affinity label on the cell surface.

The present invention discloses a bacterial cellulose-based biosensor, which comprises bacterial cellulose and a cell presenting a CBM on the surface. The CBM is a cellulose-binding module that specifically binds to crystalline region of cellulose. The cell is attached to the bacterial cellulose through the CBM.

The cellulose-binding module(CBM) is CBM2a.

The cell is a recombinant strain expressing the CBM by using pETDuet-tac as a vector. The pETDuet-tac is a vector obtained by replacing two T7 promoters on the vector pETDuet by two tac promoters, that is, an upstream first tac promoter and a downstream second tac promoter. The pETDuet-tac comprises a gene encoding a fluorescent protein downstream of the first tac promoter and a gene encoding the CBM presented on the surface downstream of the second tac promoter.

The first tac promoter is replaced by a promoter inducible by a substance to be tested, which affects the transcription of the downstream fluorescent protein coding gene in the presence of the target compound.

Further, the CBM is presented on the surface by fusing the CBM to ankyrin presented on the surface.

Further, the gene encoding CBM2a presented on the surface has a sequence as shown in SEQ ID NO. 1, which is specifically:

5′-ATGAAGGCGACCAAACTGGTGCTGGGTGCGGTTATTCTGGGCAGCA
CCCTGCTGGCGGGTTGCAGCAGCAACGCGAAAATCGACCAGGGCATTAA
CCCGTACGTGGGTTTCGAAATGGGCTATGATTGGCTGGGTCGTATGCCG
TACAAGGGTAGCGTGGAGAACGGCGCGTATAAAGCGCAGGGTGTTCAAC
TGACCGCGAAGCTGGGCTACCCGATCACCGACGATCTGGACATTTATAC
CCGTCTGGGTGGCATGGTGTGGCGTGCGGACACCAAGAGCAACGTTTAC
GGTAAAAACCACGATACCGGCGTGAGCCCGGTTTTTGCGGGTGGCGTGG
AGTATGCGATCACCCCGGAAATTGCGACCCGTCTGGAGTATCAATGGAC
CAACAACATCGGTGACGCGCACACCATTGGCACCCGTCCGGATAACGGT
ATTCCGGGCGCTAGCTCCGGTCCGGCCGGGTGCCAGGTGCTGTGGGGCG
TCAACCAGTGGAACACCGGCTTCACCGCGAACGTCACCGTGAAGAACAC
GTCCTCCGCTCCGGTCGACGGCTGGACGCTCACGTTCAGCTTCCCGTCC
GGCCAGCAGGTCACCCAGGCGTGGAGCTCGACGGTCACGCAGTCCGGCT
CGGCCGTGACGGTCCGCAACGCCCCGTGGAACGGCTCGATCCCGGCGGG
CGGCACCGCGCAGTTCGGCTTCAACGGCTCGCACACGGGCACCAACGCC
GCGCCGACGGCGTTCTCGCTCAACGGCACGCCCTGCACGGTCGGCCATC
ACCATCATCACCACTGA-3′.

Further, the bacterial cellulose can be spherical, flaky, rod-shaped or in other various forms, for detection in different scenarios.

A method for constructing the bacterial cellulose-based biosensor comprises the following step: co-incubating cells presenting the CBMs on the surface with bacterial cellulose. The method comprises specifically:
(1) ligating a gene encoding the CBM presented on the surface to a vector, and transforming the vector into a host cell to obtain a recombinant cell; and
(2) inoculating the recombinant cells obtained Step (1) into a culture medium, incubating, adding a transcription inducer and bacterial cellulose when OD₆₀₀ is 0.6-0.8, and further incubating for 10-12 hrs to obtain the bacterial cellulose-based biosensor.

Further, the cell is a recombinant strain, with E. coli as a host, and using pETDuet-tac as a vector. The pETDuet-tac is a vector obtained by replacing two T7 promoters on the vector pETDuet by two tac promoters, that is, an upstream first tac promoter and a downstream second tac promoter. The pETDuet-tac comprises a gene encoding a fluorescent protein downstream of the first tac promoter and a gene encoding the CBM presented on the surface downstream of the second tac promoter.

Further, to detect various substances, the first tac promoter can be replaced by a specific promoter that affects the transcription of the downstream fluorescent protein coding gene in the presence of the target compound, for example, a promoter inducible by L-arabinose (a nucleic acid fragment containing L-arabinose promoter and AraC), a promoter inducible by a nitro compound, or a promoter inducible by a heavy metal.

Further, the promoter inducible by L-arabinose (a nucleic acid fragment containing L-arabinose promoter and AraC) has a sequence as shown in SEQ ID NO. 3, which is specifically:

5′-TTATGACAACTTGACGGCTACATCATTCACTTTTTCTTCACAACCG
GCACGGAACTCGCTCGGGCTGGCCCCGGTGCATTTTTTAAATACCCGCG
AGAAGTAGAGTTGATCGTCAAAACCAACATTGCGACCGACGGTGGCGAT
AGGCATCCGGGTGGTGCTCAAAAGCAGCTTCGCCTGGCTGATACGTTGG
TCCTCGCGCCAGCTTAAGACGCTAATCCCTAACTGCTGGCGGAAAAGAT
GTGACAGACGCGACGGCGACAAGCAAACATGCTGTGCGACGCTGGCGAT
ATCAAAATTGCTGTCTGCCAGGTGATCGCTGATGTACTGACAAGCCTCG
CGTACCCGATTATCCATCGGTGGATGGAGCGACTCGTTAATCGCTTCCA
TGTGCCGCAGTAACAATTGCTCAAGCAGATTTATCGCCAGCAGCTCCGA
ATAGCGCCCTTCCCCTTGCCCGGCGTTAATGATTTGCCCAAACAGGTCG
CTGAAATGCGGCTGGTGCGCTTCATCCGGGCGAAAGAACCCCGTATTGG
CAAATATTGACGGCCAGTTAAGCCATTCATGCCAGTAGGCGCGCGGACG
AAAGTAAACCCACTGGTGATACCATTCGCGAGCCTCCGGATGACGACCG
TAGTGATGAATCTCTCCTGGCGGGAACAGCAAAATATCACCCGGTCGGC
AAACAAATTCTCGTCCCTGATTTTTCACCACCCCCTGACCGCGAATGGT
GAGATTGAGAATATAACCTTTCATTCCCAGCGGTCGGTCGATAAAAAAA
TCGAGATAACCGTTGGCCTCAATCGGCGTTAAACCCGCCACCAGATGGG
CATTAAACGAGTATCCCGGCAGCAGGGGATCATTTTGCGCTTCAGCCAT
ACTTTTCATACTCCCGCCATTCAGAGAAGAAACCAATTGTCCATATTGC
ATCAGACATTGCCGTCACTGCGTCTTTTACTGGCTCTTCTCGCTAACCA
AACCGGTAACCCCGCTTATTAAAAGCATTCTGTAACAAAGCGGGACCAA
AGCCATGACAAAAACGCGTAACAAAAGTGTCTATAATCACGGCAGAAAA
GTCCACATTGATTATTTGCACGGCGTCACACTTTGCTATGCCATAGCAT
TTTTATCCATAAGATTAGCGGATCCTACCTGACGCTTTTTATCGCAACT
CTCTACTGTTTCTCCAT-3′.

Further, the fluorescent protein includes, but is not limited to, green fluorescent protein, red fluorescent protein, cyan fluorescent protein, and others. For example, the gene sequence is as shown in SEQ ID NO. 2, which is specifically:

5′-ATGTCAAAAGGCGAAGAACTGTTTACCGGCGTTGTTCCGATTCTGG
TTGAACTGGATGGTGATGTGAATGGCCATAAATTTAGCGTGTCAGGCGA
AGGCGAAGGTGATGCCACCTATGGCAAACTGACCCTGAAATTTATTTGT
ACCACCGGCAAACTGCCGGTTCCGTGGCCGACCTTAGTGACCACCCTGA
CCTATGGTGTGCAGTGTTTTAGTCGCTATCCGGATCACATGAAACAGCA
TGATTTTTTTAAATCTGCAATGCCGGAAGGCTATGTGCAGGAACGCACC
ATTTTTTTTAAAGATGATGGTAATTATAAAACCCGCGCCGAAGTTAAAT
TTGAAGGTGATACCTTAGTTAATCGTATTGAACTGAAAGGCATTGATTT
TAAAGAAGATGGCAATATTCTGGGCCATAAACTGGAATATAATTATAAT
AGTCATAATGTGTATATTATGGCCGATAAACAGAAAAATGGTATTAAAG
TTAATTTTAAAATTCGTCATAATATTGAAGATGGCTCAGTGCAGTTAGC
CGATCATTATCAGCAGAATACCCCGATTGGTGATGGTCCGGTTCTGCTG
CCGGATAATCATTATCTGTCTACCCAGAGCGCCCTGAGCAAAGATCCGA
ATGAAAAACGCGATCACATGGTTCTGCTGGAATTTGTGACCGCAGCAGG
TATTACCCTGGGCATGGATGAACTGTATAAATAA-3′.

A method for constructing a biosensor inducible by a test substance comprises the following steps:
(1) ligating a gene encoding the CBM presented on the surface and a gene encoding a fluorescent protein to the vector pETDuet-tac, to obtain the vector pETDuet-tac-EGFP-CBM, wherein the gene encoding the fluorescent protein is located downstream of the first tac promoter, and the gene coding the CBM presented on the surface is located downstream of the second tac promoter; and
(2) using the constructed vector pETDuet-tac-EGFP-CBM as a template, replacing the first tac promoter by a promoter inducible by a substance to be tested, transforming the vector into a host strain, and co-incubating the recombinant strain with the bacterial cellulose to obtain the biosensor.

Further, in Step (2), the first tac promoter is deleted by inverse PCR amplification using an upstream promoter as shown in SEQ ID NO. 4 and a downstream promoter as shown in SEQ ID NO. 5. The sequences of the upstream primer and the downstream primer are specifically as follows:

SEQ ID NO. 4:
5′-CAATCGATCTCGATCCTCTACG-3′;
SEQ ID NO. 5:
5′-TTTCACACAGGAAACAGTATC-3′.

The biosensor of the present invention is widely used in the detection of monosaccharides, explosive molecules and heavy metals. Specifically, the biosensor of the present invention is mixed with a sample to be detected, and incubated for 3-60 hrs. Then, the fluorescence intensity is detected, to realize the detection of the test substance.

By virtue of the above technical solutions, the present invention has at least the following advantages.
(1) According to the present invention, by presenting CBM on the cell surface, the efficient and specific immobilization of cells on the BC substrate can be realized without any modification of the BC substrate. After an external shearing force is continuously applied to the BC carrier loaded with whole cells for 60 hrs, the cells presenting CBM on the surface can still be closely bound to the BC carrier.
(2) In the present invention, the strategy of simultaneous presenting and immobilization is adopted, to realize simple and rapid loading of whole cells.
(3) The bacterial cellulose (BC)-based biosensor provided in the present invention has great potential in the field of substance detection.

The above description is only a summary of the technical solutions of the present invention. To make the technical means of the present invention clearer and implementable in accordance with the disclosure of the specification, the preferred embodiments of the present invention will be described in detail with reference to the accompanying drawings.

### BRIEF DESCRIPTION OF THE DRAWINGS

To make the disclosure of the present invention more comprehensible, the present invention will be further described in detail by way of specific embodiments of the present invention with reference the accompanying drawings, in which

FIG. 1 is an SDS-PAGE electrophoretogram, in which 1 indicates a soluble protein derived from recombinant E. coli BL21(DE3) containing the plasmid pETDuet-tac-CBM2a, and 2 indicates a soluble protein derived from native E. coli BL21(DE3);

FIG. 2 is an immunofluorescence micrograph of recombinant E. coli with CBM2a presented on its surface;

FIG. 3 is an SEM image showing the surface morphology of a bacterial cellulose (BC) carrier loaded with E. coli cells presenting CBM2a on the surface;

FIG. 4 is a fluorescence image of a flaky and a spherical BC-based fluorescence biosensor for detecting L-arabinose (320 mg/L), respectively;

FIG. 5 shows the relationship between L-arabinose concentration and fluorescence intensity; and

FIG. 6 shows a fluorescence image of a flaky and a spherical BC-based fluorescence biosensor for detecting L-arabinose in soil.

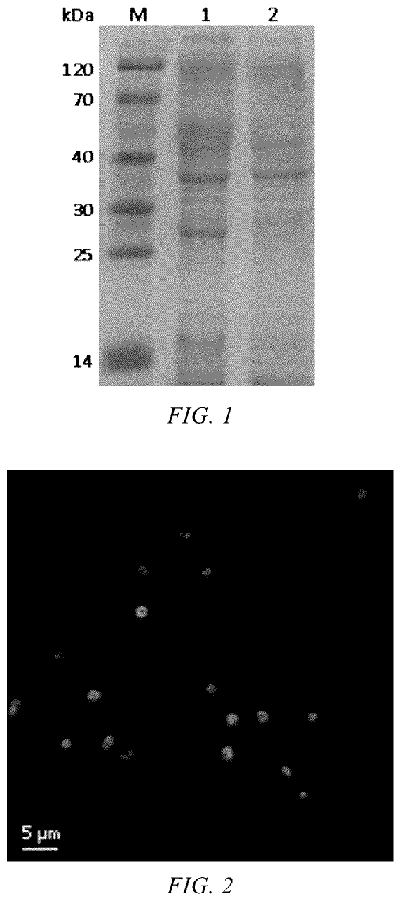

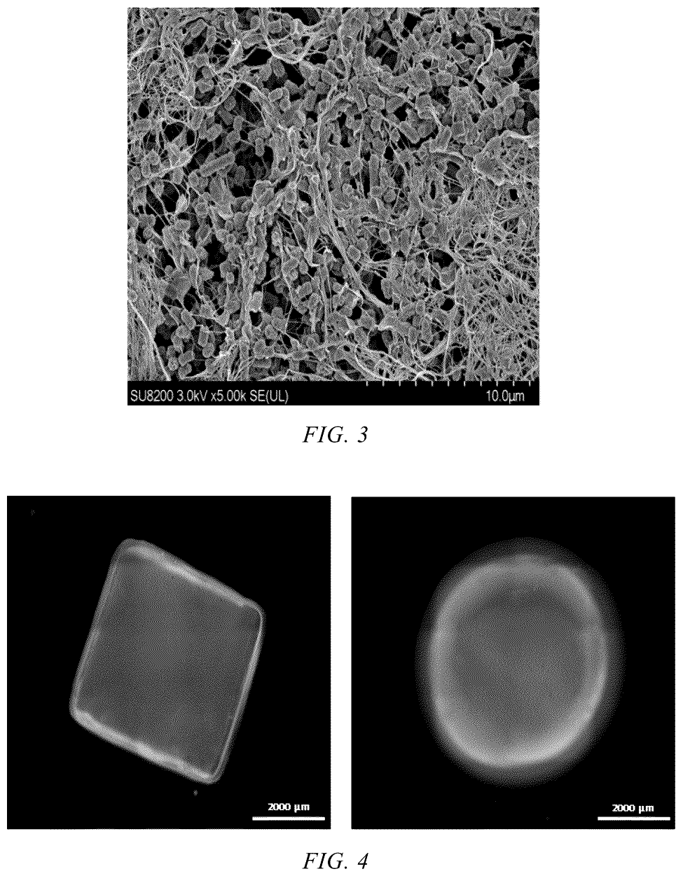

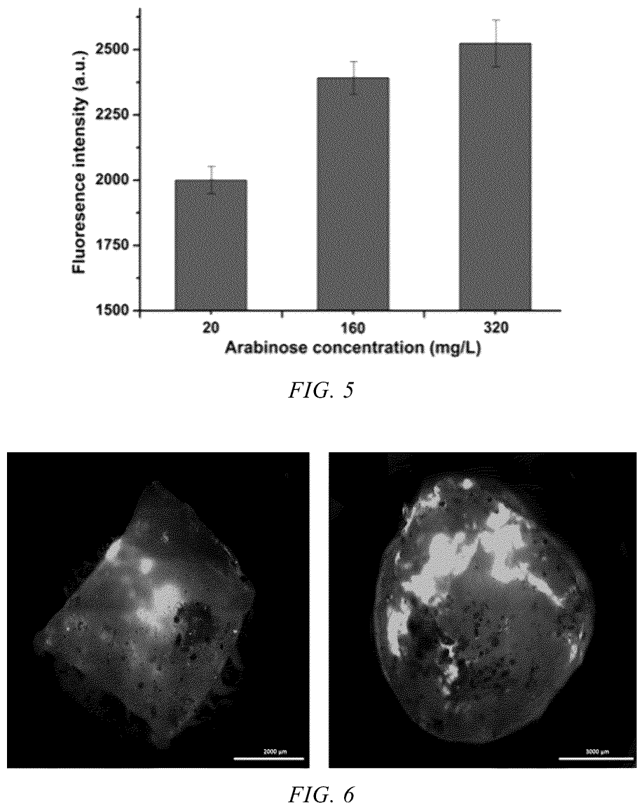

### DETAILED DESCRIPTION OF THE PREFERRED EMBODIMENTS

The present invention will be further described below with reference to the accompanying drawings and specific examples, so that those skilled in the art can better understand and implement the present invention; however, the present invention is not limited thereto.

The methods given in examples below are all conventional methods, unless it is otherwise stated. The materials and reagents used are all commercially available, unless it is otherwise stated.

### Example 1

(1) By using the PCR technology, the gene, as shown in SEQ ID NO. 1, encoding CBM2a presented on the surface of E. coli BL21(DE3) was inserted into the plasmid pETDuet-tac (using pETDuet as a template, in which two T7 promoter were replaced by two tac promoters, and which were preserved in the laboratory). The plasmid was enzymatically cleaved with the endonucleases NdeI and KpnI, purified by using a PCR purification kit, and recovered. Then, the sequence as shown in SEQ ID NO. 1 was ligated to the vector pETDuet-tac at 16° C. overnight by using T4 ligase. The ligated product was transformed into competent E. coli DH5a cells, and the vector pETDuet-tac-CBM2a was obtained after verification by colony PCR and sequencing. The pETDuet-tac-CBM2a was transformed into the host strain E. coli BL21(DE3), to obtain recombinant cells presenting CBM2a on the surface.
(2) A single colony of Acetobacter xylinum was statically cultured for 2 days in 50 mL of HS medium (containing glucose 40 g/L, yeast extract 5 g/L, peptone 5 g/L, Na₂HPO₄ 2.7 g/L, and acetic acid 1.5 g/L) at 30° C., to prepare a seed culture. 5 mL of the seed culture was transferred to 100 mL of HS medium and cultured statically at 30° C. for 15 days, to prepare a bacterial cellulose (BC) membrane.
(3) A single colony of recombinant E. coli BL21(DE3) cells presenting CBM2a on the surface obtained in Step (1) was picked up and seeded into 5 mL of Luria-Bertani broth (LB) medium containing 100 μg/mL Amp, and cultured at 37° C. with shaking at 200 rpm for 8-12 hrs. 1 mL of the cell culture was seeded into a 500 mL shake flask containing 100 mL culture medium, and cultured at 37° C. with shaking at 200 rpm. When OD₆₀₀ reached 0.6-0.8, IPTG (with a final concentration of 0.25 mM) and BC matrix (obtained in Step 2) were added, and the cells were cultured for 12 hrs at 25° C. with shaking at 150 rpm, to obtain a BC carrier loaded with recombinant E. coli cells. Then, the BC carrier was thoroughly washed with 50 mM potassium phosphate buffer (pH 7.0) to obtain a bacterial cellulose (BC)-based biosensor.
(4) To determine the ability of E. coli presenting CBM2a to bind to BC, the cells released from the BC carrier was determined by measuring the fluorescence intensity and OD₆₀₀ of the solution incubated with BC carrier. BC loaded with E. coli cells was cultured in 50 mM potassium phosphate buffer (pH 7.0) in a rotary hybridization system at 4° C. Then, samples were collected from the solution at various time points for fluorescence and OD₆₀₀ measurement. Using a 96-well cell culture plate, the fluorescence intensity of escaped E. coli cells and the duration of OD₆₀₀ were measured on cytation 5 imaging reader. The fluorescence of EGFP expressed in cells was characterized by using excitation light with λex=480 nm and emission light with λem=520 nm.

The result shows that after an external shearing force is continuously applied to the BC carrier loaded with cells for 60 hrs, the cells presenting CBM on the surface can still be closely bound to the BC carrier.

### Example 2

(1) By using the PCR technology, a gene encoding CBM presented on the surface of E. coli BL21(DE3) that specifically binds to crystalline region of cellulose could be inserted into the plasmid pETDuet-tac. The plasmid was enzymatically cleaved with the endonucleases NdeI and KpnI, purified by using a PCR purification kit, and recovered. The gene encoding CBM presented on the surface of E. coli BL21(DE3) that specifically binds to the crystalline region of cellulose was ligated to the vector pETDuet-tac at 16° C. overnight by T4 ligase. The ligated product was transformed into competent E. coli DH5a cells, and the vector pETDuet-tac-CBM2a was obtained after verification by colony PCR and sequencing. The pETDuet-tac-CBM2a was transformed into the host strain E. coli BL21(DE3), to obtain recombinant cells presenting CBM2a on the surface.

(2) A single colony of Acetobacter xylinum was statically cultured for 2 days in 50 mL of HS medium (containing glucose 40 g/L, yeast extract 5 g/L, peptone 5 g/L, Na₂HPO₄ 2.7 g/L, and acetic acid 1.5 g/L) at 30° C., to prepare a seed culture. 10 mL of the seed culture was then transferred to 100 mL of HS medium and cultured statically at 30° C. for 15 days, to prepare a BC membrane.

(3) A single colony of recombinant E. coli BL21(DE3) cells presenting CBM2a that specifically binds to crystalline region of cellulose on the surface obtained in Step (1) were picked up and seeded into 5 mL of Luria-Bertani broth (LB) medium containing 100 μg/mL Amp, and cultured at 37° C. with shaking at 200 rpm for 8-12 hrs. 1 mL of the cell culture was seeded into a 500 mL shake flask containing 100 mL culture medium, and cultured at 37° C. with shaking at 200 rpm. When OD₆₀₀ reached 0.6-0.8, IPTG (with a final concentration of 0.25 mM) and BC matrix (obtained in Step 2) were added, and the cells were cultured for 12 hrs at 25° C. with shaking at 150 rpm, to obtain a BC carrier loaded with recombinant E. coli cells. Then, the BC carrier was thoroughly washed with 50 mM potassium phosphate buffer (pH 7.0) to obtain a BC-based biosensor.

### Example 3

(1) By using the PCR technology, the gene, as shown in SEQ ID NO. 1, encoding CBM2a presented on the surface of E. coli BL21(DE3) could be inserted into the plasmid pETDuet-tac (using pETDuet as a template, in which two T7 promoter were replaced by two tac promoters, and which were preserved in the laboratory). The plasmid was enzymatically cleaved with the endonucleases NdeI and KpnI, purified by using a PCR purification kit, and recovered. Then, the sequence as shown in SEQ ID NO. 1 was ligated to the vector pETDuet-tac at 16° C. overnight by using T4 ligase. The ligated product was transformed into competent E. coli DH5a cells, and the vector pETDuet-tac-CBM2a was obtained after verification by colony PCR and sequencing. Using pETDuet-tac-CBM2a as a template, the sites were enzymatically cleaved with NcoI and EcoRI, and the above enzymatic cleavage and ligation steps were repeated to insert the green fluorescent protein coding gene as shown in SEQ ID NO. 2 into pETDuet-tac-CBM2a. The ligated product was transformed into competent E. coli DH5a, and the vector pETDuet-tac-EGFP-CBM2a was obtained after verification by colony PCR and sequencing. The pETDuet-tac-EGFP-CBM2a was transformed into the host strain E. coli BL21(DE3), to obtain recombinant chassis fluorescent cell.

(2) A single colony of Acetobacter xylinum was statically cultured for 2 days in 50 mL of HS medium (containing glucose 40 g/L, yeast extract 5 g/L, peptone 5 g/L, Na₂HPO₄ 2.7 g/L, and acetic acid 1.5 g/L) at 30° C., to prepare a seed culture.

10 mL of the seed culture and 220 mL of HS medium were transferred into a 250 mL flask, and incubated for 5 days at 30° C. with shaking at 150 rpm, to prepare spherical BC. 10 mL of the seed culture and 100 ml of HS medium were poured into a 250 mL flask, and statically incubated for 15 days at 30° C., to prepare flaky BC. 10 mL of the seed culture and 100 ml of HS medium were mixed and then injected into a silica tube, and then statically incubated for 10 days at 30° C., to prepare tubular BC.

(3) A single colony of fluorescent E. coli BL21(DE3) chassis cells obtained in Step (1) was picked up and seeded into 5 mL of Luria-Bertani broth (LB) medium containing 100 μg/mL Amp, and cultured at 37° C. with shaking at 200 rpm for 8-12 hrs. 1 mL of the cell culture was seeded into a 500 mL shake flask containing 100 mL culture medium, and cultured at 37° C. with shaking at 200 rpm. When OD₆₀₀ reached 0.6-0.8, IPTG (with a final concentration of 0.25 mM) and BC matrix (obtained in Step 2) were added, and the cells were cultured for 12 hrs at 25° C. with shaking at 150 rpm, to obtain a flaky and a spherical BC carrier loaded with recombinant E. coli cells respectively, depending on the shape of the BC matrix. Then, the BC carrier was thoroughly washed with 50 mM potassium phosphate buffer (pH 7.0).

### Example 4

Using the vector pETDuet-tac-EGFP-CBM2a constructed in Example 1 as a vector, inverse PCR amplification was using primers shown below, to delete the tac promoter (first tac promoter) regulating green fluorescent protein:

primer 1 (SEQ ID NO. 6):
5′-CAATCGATCTCGATCCTCTACG-3′;
primer 2 (SEQ ID NO. 7):
5′-TTTCACACAGGAAACAGTATC-3′.

In addition, using plasmid pCAS as a template, PCR amplification was performed with the following primers to obtain a nucleic acid fragment as shown in SEQ ID NO. 3 and comprising P_{araBAD} and AraC:

primer 3 (SEQ ID NO. 8):
5′-TAGAGGATCGAGATCGATTGTTATGACAACTTGACGGCTACA
TC-3′;
primer 4 (SEQ ID NO. 9):
5′-GATACTGTTTCCTGTGTGAAAATGGAGAAACAGTAGAGAGTTG
CG-3′.

Then, the fragment was ligated by using ClonExpress II One Step Cloning Kit (purchased from Vazyme), and the tac promoter regulating green fluorescent protein in the vector pETDuet-tac-EGFP-CBM2a was replaced by the nucleic acid fragment as shown in SEQ ID NO. 3 and comprising arabinose promoter (P_{araBAD}) and AraC. The ligated product was transformed into competent E. coli DH5α cells, and the vector pETDuet-araBAD-EGFP-CBM2a was obtained after verification by colony PCR and sequencing. The recombinant plasmid pETDuet-araBAD-EGFP-CBM2a was transformed into the host strain E. coli BL21(DE3), and the cells were attached to BC according to the method described in Example 1 to obtain an L-arabinose-inducible fluorescence biosensor.

### Example 5

(1) The L-arabinose-inducible fluorescence biosensor was used to detect L-arabinose in aqueous solution. The specific method was as follows. The L-arabinose-inducible fluorescence biosensor prepared above was immersed respectively in L-arabinose solutions with various concentrations, and incubated at room temperature for 5 hrs. Then the fluorescence intensity was measured and fluorescence imaging was carried out to detect L-arabinose in the solutions.

The L-arabinose solution was prepared by diluting an L-arabinose stock solution serially with water to give a final concentration of 20 mg/L, 160 mg/L and 320 mg/L respectively.

(2) The L-arabinose-inducible fluorescence biosensor was used to detect L-arabinose in soil. The specific method was as follows. A required soil sample was prepared by mixing L-arabinose with soil at a dosage of 2.4 g L-arabinose/Kg soil. Then, the fluorescence biosensor was buried in the sample, and incubated at 37° C. for 24 hrs. Then fluorescence imaging was performed to detect L-arabinose in the soil.

The results show that the lowest detectable concentration is 15 mg/L.

### Example 6

(1) The vector pETDuet-tac-EGFP-CBM2a constructed in Example 1 was used as a vector, in which the tac promoter regulating green fluorescent protein in the vector pETDuet-tac-EGFP-CBM2a was replaced by the yqjF promoter. The vector pETDuet-yqjF-EGFP-CBM2a was obtained. The recombinant plasmid pETDuet-yqjF-EGFP-CBM2a was transformed into the host strain E. coli BL21(DE3), and the cells were attached to BC according to the method described in Example 3 to obtain a 2,4-dinitrotoluene (2,4-DNT) inducible fluorescence biosensor.

(2) The 2,4-DNT inducible fluorescence biosensor was used to detect 2,4-DNT in aqueous solution. The specific method was as follows. The 2,4-DNT inducible fluorescence biosensor prepared above was immersed respectively in 2,4-DNT solutions with various concentrations, and incubated at room temperature for 12 hrs. Then the fluorescence intensity was measured and fluorescence imaging was carried out to detect 2,4-DNT in the solutions.

The 2,4-DNT solution was prepared by diluting an 2,4-DNT stock solution serially with water to give a final concentration of 5 mg/L, 10 mg/L and 20 mg/L respectively.

(3) The 2,4-DNT inducible fluorescence biosensor was used to detect 2,4-DNT in soil. The specific method was as follows. A required soil sample was prepared by mixing 2,4-DNT with soil at a dosage of 0.24 g 2,4-DNT/Kg soil. Then, the fluorescence biosensor was buried in the sample, and incubated at 37° C. for 24 hrs. Then fluorescence imaging was performed to detect 2,4-DNT in the soil.

The results show that the lowest detectable concentration is 4 mg/L.

### Example 7

(1) The vector pETDuet-tac-EGFP-CBM2a constructed in Example 1 was used as a template, in which the tac promoter relating green fluorescent protein in the vector pETDuet-tac-EGFP-CBM2a was replaced by the znt promoter (comprising zntA promoter and zntR nucleic acid sequence). The vector pETDuet-znt-EGFP-CBM2a was obtained. The recombinant plasmid pETDuet-CBM2a-znt-EGFP was transformed into the host strain E. coli BL21(DE3), and the cells were attached to BC according to the method described in Example 3 to obtain a heavy metal-inducible fluorescence biosensor.

(2) The heavy metal-inducible fluorescence biosensor was used to detect a heavy metal in aqueous solution. The specific method was as follows. The heavy metal-inducible fluorescence biosensor prepared above was immersed respectively in heavy metal solutions with various concentrations, and incubated at room temperature for 24 hrs. Then the fluorescence intensity was measured and fluorescence imaging was carried out to detect a heavy metal in the solutions.

The heavy metal solution comprises Zn²⁺, Cd²⁺, or Hg²⁺. The preparation method was as follows. A Zn²⁺ stock solution was serially diluted with water to a final concentration of 20 mg/L, 100 mg/L and 300 mg/L respectively. A Cd²⁺ stock solution was serially diluted with water to a final concentration of 0.5 mg/L, 2.0 mg/L and 4.0 mg/L respectively. A Hg²⁺ stock solution was serially diluted with water to a final concentration of 0.004 mg/L, 0.016 mg/L and 0.06 mg/L, respectively.

(3) The heavy metal-inducible fluorescence biosensor was used to detect a heavy metal in soil. The specific method was as follows. A required soil sample was prepared by mixing a heavy metal respectively with soil at a dosage of 0.3 g Zn²/Kg soil, 4 mg Cd²⁺/Kg soil and 0.06 mg Hg²⁺/Kg. Then, the fluorescence biosensor was buried in the sample, and incubated at 37° C. for 24 hrs. Then fluorescence imaging was performed to detect a heavy metal in the soil.

The results show that the lowest detectable concentrations are Zn²⁺ 6 mg/L, Cd²⁺ 0.05 mg/L, and Hg²⁺ 0.004 mg/L.

### Comparative Example 1

(1) The PCR technology was used, the vector pETDuet-tac-EGFP-CBM2a constructed in Example 1 was used as a template, and the second tac promoter was replaced by the T7 promoter. The vector pETDuet-tac-EGFP-T7-CBM2a was obtained. Then an L-arabinose-inducible fluorescence biosensor was prepared following the steps in Example 4.

(2) To compare the expression of CBM2a fusion protein presented on the surface in the presence of different promoters, SDS-PAGE was used to detect the intracellular soluble protein, and the ability of E. coli cells presenting CBM2a to bind to BC was monitored by determining the fluorescence intensity and OD₆₀₀ of the solution incubated with the BC carrier according to Step (4) in Example 1. Then, L-arabinose in aqueous solution was detected by the L-arabinose-inducible fluorescence biosensor following the steps in Example 5.

The results show that overexpressed soluble proteins are present in both cells with different promoters, and the intracellular protein content in cells with T7 promoter was about 20% higher than that in cells with tac promoter. The experiment of cells' binding ability to BC shows that both cells can bind to the BC carrier closely, and the binding performances are basically the same. However, the higher overexpression of the protein requires higher consumption of the resources and energy in the cell. The detection sensitivity of the L-arabinose-inducible fluorescence biosensor containing T7 promoter decreases (by a factor of 2.1), and the lowest detectable concentration of L-arabinose is 32 mg/L.

### Comparative Example 2

(1) The PCR technology was used, the vector pETDuet-tac-EGFP-CBM2a constructed in Example 1 was used as a template, and CBM2a was replaced by CBM44. The vector pETDuet-tac-EGFP-CBM44 was obtained.

Specific gene sequence of CBM44 (SEQ ID NO. 10):

5′-AAATTCAATTTTGAAGATGGAACACTAGGGGGCTTTACCACCTC
TGGCACCAATGCGACCGGTGTTGTGGTGAACACCACTGAAAAAGCGT
TTAAGGGTGAACGTGGTCTGAAGTGGACCGTCACGTCCGAGGGCGAG
GGCACCGCTGAGCTCAAGCTGGACGGCGGTACGATCGTGGTGCCGGG
TACGACGATGACATTCCGCATTTGGATTCCGAGCGGCGCGCCAATCG
CCGCAATTCAACCGTACATTATGCCGCACACCCCGGATTGGAGCGAA
GTTCTGTGGAACAGCACCTGGAAAGGTTATACCATGGTCAAAACTGA
CGATTGGAACGAGATCACCTTGACCCTGCCGGAAGATGTTGACCCGA
CGTGGCCGCAGCAAATGGGTATTCAGGTTCAGACCATCGACGAAGGT
GAGTTCACCATCTACGTGGATGCGATCGACTGGTGA-3′.

The pETDuet-tac-EGFP-CBM44 was transformed into the host strain E. coli BL21(DE3), to obtain recombinant cells presenting CBM44 on the surface. A BC-based biosensor was obtained following the method in Example 1.

(2) To determine the ability of E. coli presenting CBM44 to bind to BC, the cells released from the BC carrier was determined by measuring the fluorescence intensity and OD₆₀₀ of the solution incubated with BC carrier following the method in Example 1(4).

The result shows that after an external shearing force is continuously applied to the BC carrier loaded with cells for 60 hrs, the cells presenting CBM44 on the surface escape, and cannot be closely bound to the BC carrier.
(3) By using the constructed vector pETDuet-tac-EGFP-CBM44 as a template, the tac promoter was replaced by a nucleic acid fragment comprising arabinose promoter (P_{araBAD}) and AraC following the method in Example 4. Finally, an L-arabinose-inducible fluorescence biosensor bound with cells presenting CBM44 on the surface was obtained. Then, L-arabinose in aqueous solution and soil was detected respectively according to the method in Example 5.

The results show that due to cell escape, the detection sensitivity of the L-arabinose-inducible fluorescence biosensor bound with cells presenting CBM44 on the surface decreases greatly. For example, L-arabinose could not be detected normally at 20 mg/L, and the accuracy was less than 60% at 320 mg/L.

Apparently, the above-described embodiments are merely examples provided for clarity of description, and are not intended to limit the implementations of the present invention. Other variations or changes can be made by those skilled in the art based on the above description. The embodiments are not exhaustive herein. Obvious variations or changes derived therefrom also fall within the protection scope of the present invention.

What is claimed is:

1. A bacterial cellulose-based biosensor, comprising bacterial cellulose and a cell presenting a cellulose-binding module on the surface, wherein the cellulose-binding module specifically binds to crystalline region of cellulose, and the cell is attached to the bacterial cellulose through the cellulose-binding module, wherein
the cellulose-binding module is CBM2a;
the cell is a recombinant strain expressing the cellulose-binding module by using pETDuet-tac as a vector, wherein the pETDuet-tac is a vector obtained by replacing two T7 promoters on the vector pETDuet by two tac promoters, that is, an upstream first tac promoter and a downstream second tac promoter; and the pETDuet-tac comprises, a gene encoding a fluorescent protein downstream of the first tac promoter and a gene encoding the cellulose-binding module presented on the surface downstream of the second tac promoter;
the first tac promoter is replaced by a promoter inducible by a substance to be tested, which affects the transcription of the downstream fluorescent protein coding gene in the presence of the target compound.

2. The biosensor according to claim 1, wherein the gene encoding CBM2a presented on the surface has a sequence as shown in SEQ ID NO. 1.

3. The biosensor according to claim 1, wherein the promoter inducible by a substance to be tested is a promoter inducible by L-arabinose, a promoter inducible by a nitro compound, or a promoter inducible by a heavy metal.

4. The biosensor according to claim 3, wherein the promoter inducible by L-arabinose has a nucleotide sequence as shown in SEQ ID NO. 3.

5. The biosensor according to claim 1, wherein the recombinant strain is a recombinant E. coli strain.

6. A method for constructing a biosensor according to claim 1, comprising steps of:
(1) ligating a gene encoding the cellulose-binding module presented on the surface and a gene encoding a fluorescent protein to the vector pETDuet-tac, to obtain the vector pETDuet-tac-EGFP-CBM, wherein the gene encoding the fluorescent protein is located downstream of the first tac promoter, and the gene coding the cellulose-binding module presented on the surface is located downstream of the second tac promoter; and the cellulose-binding module is CBM2a; and
(2) using the constructed vector pETDuet-tac-EGFP-CBM as a template, replacing the first tac promoter by a promoter inducible by a substance to be tested, transforming the vector into a host strain to obtain a recombinant strain, and co-incubating the recombinant strain with the bacterial cellulose to obtain the biosensor.

7. Use of the biosensor according to claim 1 in the detection of a substance.

8. The use according to claim 7, wherein the substance comprises a monosaccharide, an explosive molecule or a heavy metal.

9. The use according to claim 7, wherein the detection of a substance comprises steps of: mixing and incubating the biosensor with a sample to be tested, and then detecting the fluorescence intensity.


SEQUENCE LISTING (10 sequences)

SEQ ID NO: 1 - DNA, 798 bp, Synthetic construct
  source 1..798: mol_type = other DNA
atgaaggcgaccaaactggtgctgggtgcggttattctgggcagcaccctgctggcgggttgcagcagcaacgcgaaaatcgaccagggcattaacccgtacgtgggtttcgaaatgggctatgattggctgggtcgtatgccgtacaagggtagcgtggagaacggcgcgtataaagcgcagggtgttcaactgaccgcgaagctgggctacccgatcaccgacgatctggacatttatacccgtctgggtggcatggtgtggcgtgcggacaccaagagcaacgtttacggtaaaaaccacgataccggcgtgagcccggtttttgcgggtggcgtggagtatgcgatcaccccggaaattgcgacccgtctggagtatcaatggaccaacaacatcggtgacgcgcacaccattggcacccgtccggataacggtattccgggcgctagctccggtccggccgggtgccaggtgctgtggggcgtcaaccagtggaacaccggcttcaccgcgaacgtcaccgtgaagaacacgtcctccgctccggtcgacggctggacgctcacgttcagcttcccgtccggccagcaggtcacccaggcgtggagctcgacggtcacgcagtccggctcggccgtgacggtccgcaacgccccgtggaacggctcgatcccggcgggcggcaccgcgcagttcggcttcaacggctcgcacacgggcaccaacgccgcgccgacggcgttctcgctcaacggcacgccctgcacggtcggccatcaccatcatcaccactga

SEQ ID NO: 2 - DNA, 717 bp, Synthetic construct
  source 1..717: mol_type = other DNA
atgtcaaaaggcgaagaactgtttaccggcgttgttccgattctggttgaactggatggtgatgtgaatggccataaatttagcgtgtcaggcgaaggcgaaggtgatgccacctatggcaaactgaccctgaaatttatttgtaccaccggcaaactgccggttccgtggccgaccttagtgaccaccctgacctatggtgtgcagtgttttagtcgctatccggatcacatgaaacagcatgatttttttaaatctgcaatgccggaaggctatgtgcaggaacgcaccattttttttaaagatgatggtaattataaaacccgcgccgaagttaaatttgaaggtgataccttagttaatcgtattgaactgaaaggcattgattttaaagaagatggcaatattctgggccataaactggaatataattataatagtcataatgtgtatattatggccgataaacagaaaaatggtattaaagttaattttaaaattcgtcataatattgaagatggctcagtgcagttagccgatcattatcagcagaataccccgattggtgatggtccggttctgctgccggataatcattatctgtctacccagagcgccctgagcaaagatccgaatgaaaaacgcgatcacatggttctgctggaatttgtgaccgcagcaggtattaccctgggcatggatgaactgtataaataa

SEQ ID NO: 3 - DNA, 1190 bp, Synthetic construct
  source 1..1190: mol_type = other DNA
ttatgacaacttgacggctacatcattcactttttcttcacaaccggcacggaactcgctcgggctggccccggtgcattttttaaatacccgcgagaagtagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgtgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccat

SEQ ID NO: 4 - DNA, 22 bp, Synthetic construct
  source 1..22: mol_type = other DNA
caatcgatctcgatcctctacg

SEQ ID NO: 5 - DNA, 21 bp, Synthetic construct
  source 1..21: mol_type = other DNA
tttcacacaggaaacagtatc

SEQ ID NO: 6 - DNA, 22 bp, Synthetic construct
  source 1..22: mol_type = other DNA
caatcgatctcgatcctctacg

SEQ ID NO: 7 - DNA, 21 bp, Synthetic construct
  source 1..21: mol_type = other DNA
tttcacacaggaaacagtatc

SEQ ID NO: 8 - DNA, 44 bp, Synthetic construct
  source 1..44: mol_type = other DNA
tagaggatcgagatcgattgttatgacaacttgacggctacatc

SEQ ID NO: 9 - DNA, 45 bp, Synthetic construct
  source 1..45: mol_type = other DNA
gatactgtttcctgtgtgaaaatggagaaacagtagagagttgcg

SEQ ID NO: 10 - DNA, 456 bp, Synthetic construct
  source 1..456: mol_type = other DNA
aaattcaattttgaagatggaacactagggggctttaccacctctggcaccaatgcgaccggtgttgtggtgaacaccactgaaaaagcgtttaagggtgaacgtggtctgaagtggaccgtcacgtccgagggcgagggcaccgctgagctcaagctggacggcggtacgatcgtggtgccgggtacgacgatgacattccgcatttggattccgagcggcgcgccaatcgccgcaattcaaccgtacattatgccgcacaccccggattggagcgaagttctgtggaacagcacctggaaaggttataccatggtcaaaactgacgattggaacgagatcaccttgaccctgccggaagatgttgacccgacgtggccgcagcaaatgggtattcaggttcagaccatcgacgaaggtgagttcaccatctacgtggatgcgatcgactggtga